(19) United States
(12) Patent Application Publication
(10) Pub. No.: US 2006/0233664 A1
(43) Pub. Date: Oct. 19, 2006

(54) Paint testing apparatus

(76) Inventor: John Gregory Martin, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/453,783

(22) Filed: Jun. 15, 2006

Related U.S. Application Data
(63) Continuation-in-part of application No. 10/995,294, filed on Nov. 24, 2004.

(51) Int. Cl. G01N 33/00

(52) U.S. Cl. 422/58

(57) ABSTRACT

A portable paint testing apparatus has a cylindrical reservoir for containing a test solvent characterized by having an extended life. First and second porous, absorbent nibs are mounted at the ends of the reservoir, and are exposed to the solvent. One of the nibs is light in color for testing dark painted surfaces and the other nib is dark in color for testing light painted surfaces. At each end of the reservoir nib protective caps are attached to the assembly.

### CROSS-REFERENCE TO RELATED APPLICATION AND CLAIM OF PRIORITY

This application is a Continuation-In-Part of U.S. patent application Ser. No. 10/995,294, filed Nov. 22, 2004 by John G. Martin, the present inventor.

### FIELD OF INVENTION

The present invention is directed to the determination of the type of paint, either oil or water based, that has previously been applied to a surface.

### BACKGROUND OF THE INVENTION

When it is desired to paint over a previously painted surface or, alternatively, to prepare a previously painted surface to receive a new coat of the same or different type of paint, it is important to determine the type of previously applied paint, thus the preparation for repainting previously painted surfaces includes a determination of whether the previously applied paint is an oil base or a water base (latex) paint. The type of existing paint dictates the type of coating that may be applied over the existing surface and what, if any, additional preparation, treatment, or removal, or replacement of the existing coating is necessary.

It has long been the practice to apply denatured alcohol to a rag, and then to rub the rag against the painted surface. If the paint used was an oil based paint, there will be no indication of such on the rag. However, if the paint was a water based paint such as a latex paint, there will be paint residue on the rag., thus enabling the worker to identify the basic type of paint. If the painted surface is dark, a light colored rag is preferred, whereas for a light painted surface, a dark rag is preferred so that the residue, if any, may be easily seen.

There have been a number of paint testers which observe the aforementioned principles, but which are portable and do not require a rag or the equivalent thereof.

In U.S. Pat. No. 5,492,835 of Koenig there is disclosed a paint testing apparatus comprising a primary swab saturated with a predetermined substance and a secondary swab saturated with a different predetermined substance, contained in a pouch. This apparatus, in which the primary pad is adhered to the surface under test, requirements approximately fifteen minutes to produce a determination of the surface paint type. If the pad and the surface under the test are the same or similar color, the test results will be inaccurate or completely inconclusive.

In U.S. Pat. No. 6,214,291 of Kerman there is disclosed a test device for determining the type of paint present on a surface which comprises a tubular housing containing a glass vial filled with denatured alcohol. At one end of the housing is a swab, preferably of a relatively rigid absorbent felt material. The felt material is formed of two separate portions, each being a contrasting color, such as, for example, white and green. The white portion of the swab is for testing dark colored paint surfaces and the green portion is for testing light colored paint surfaces. A cap is provided for protecting the felt tip. In operation, the housing, which is of a flexible plastic material is squeezed sufficiently to break the glass vial so that the denatured alcohol wets the swab, then one portion of the two part swab is rubbed against the painted surface to be tested. If the surface is dark, the white portion of the swab is rubbed thereagainst and, as in the case of the aforementioned rag, any residue on the swab indicates a water based or latex paint. Conversely, the absence of residue indicates an oil based paint. Corresponding results are obtain with the green portion of the swab when rubbed against a light or white colored surface. The Kerman device requires the crushing of the vial and is, presumably, only usable one time, as, apparently, are the pads of the Koenig device.

### SUMMARY OF THE INVENTION

The present invention is a portable paint testing apparatus that represents an improvement over prior art devices in that it can be used more than once and gives a virtually instantaneous indication of the type of paint on a surface being tested.

The apparatus comprises a hollow, preferably cylindrical housing or barrel member of plastic or other suitable material, at each end of which is a hollow preferably plastic body inserted therein in a press fit. Each body has a tapered nose portion into which an absorbent nib which is preferably made of a relatively hard material, such as combination of a polyester fiber and a resin is inserted. Such a nib has a high degree of porosity and is highly resistant to wear. The housing member contains a liquid test material or solvent preferably of 2-butoxyethanol or ethylene glycol monobutyl ether which, in contrast to prior art alcohols, has a slower rate of evaporation leading to an extended shelf life.

Each end of the housing and body assembled has a protective plastic cap which protects the nib which is enclosed therein. The cap is dimensioned such that it snaps onto the body and is firmly retained in place. Each cap is connected to the assembly by a suitable elongated wire, chain, or filament to prevent its being lost.

The nibs at the ends of the assembly may take any of a variety of forms or shapes and are dimensioned to be a tight slip fit within the body member where they are exposed to the solvent within the barrel. By tight slip fit is meant a fit wherein the nib is insertable into or removable from the body member, yet is held firmly thereby after insertion. Further, one of the nibs is preferably dark in color for testing light colored surfaces, and the other nib is preferably light or white in color for testing dark colored surfaces.

These and other features of the present invention will be more readily apparent from the following detailed description, read in conjunction with the accompanying drawings.

### BRIEF DESCRIPTION OF THE DRAWINGS

FIG. 1 is a side elevation view of the apparatus of the invention;

FIG. 2 is a perspective view of the apparatus of the invention as it appears in its transportable form;

FIGS. 3a and 3b depict the configuration of one form of nib;

FIGS. 4a and 4b depict another form of nib;

FIGS. 5a and 5b depict another form of nib; and

FIGS. 6a and 6b depict still another, preferred configuration of the nib.

### DETAILED DESCRIPTION OF THE PREFERRED EMBODIMENT

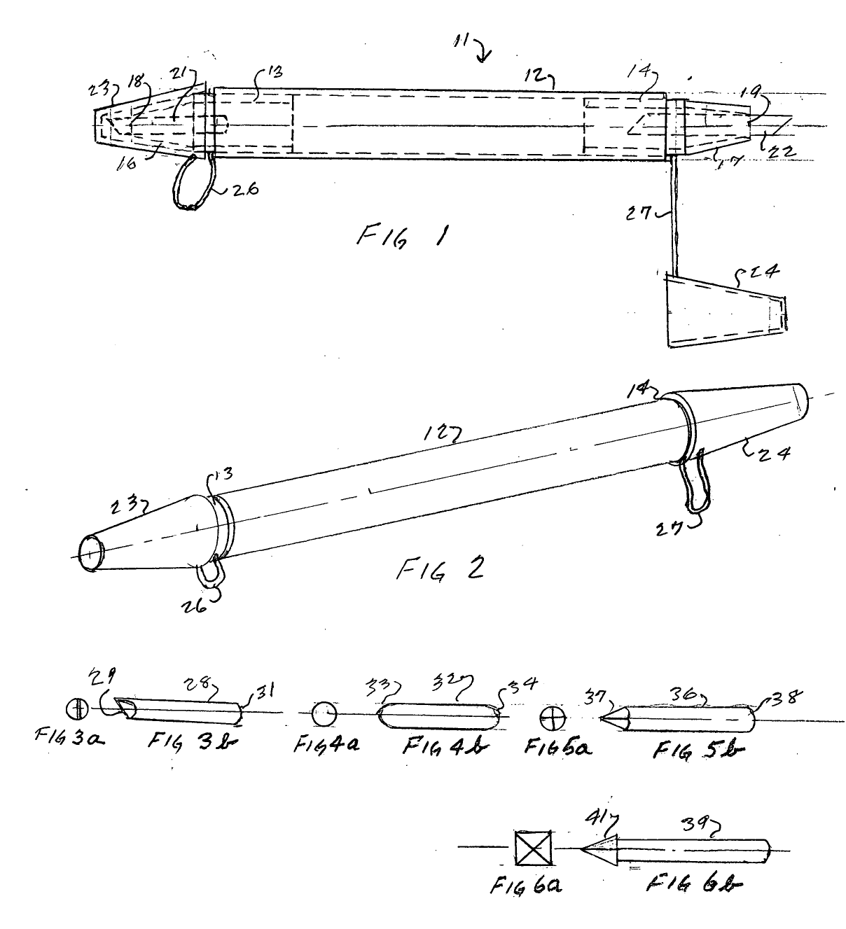

FIG. 1 depicts, in elevation, the assembled testing apparatus 11 of the invention. Apparatus 11 comprises a hollow cylindrical housing 12 which forms a reservoir for a liquid testing solvent, not shown, which preferably is a long shelf life material. Such a material is preferably 2-butoxyethanol or ethylene glycol monobutyl ether which retains its useful life over a period of time in excess of various alcohols used in the prior art. At the ends of the housing 12 are hollow body members 13 and 14 which are a press fit within housing member 12. Each body member has a tapered nose portion 16, 17, and the small end thereof has an opening 18, 19. Openings 18 and 19 are each dimensioned to receive a nib 21 and 22 respectively, which are dimensioned to be a tight slip fit within their respective holes 18 and 19. Nibs 21 and 22 are, preferably, a porous substance that is relatively hard, such as a polyester fiber-resin combinations, ensuring high resistance to wear. Other nib materials may be used, however, the combination of hardness and porosity of a polyester fiber-resin mix is to be preferred. As shown by nib 22 in FIG. 1, the nib may be double ended, or single ended, as shown by nib 21. That is, as will be discussed hereinafter, at least one end of a nib has a configured shape for rubbing against the surface under test.

When the nibs are in place within their respective body members, they are exposed to, and saturated by, the solvent within housing 12. As a consequence, it is preferable, when not in use, that the ends of the assembly are capped by tapered plastic caps 23 and 24 which enclose their respective nibs, and which may be snapped or wedged onto bodies 13 and 14. In order that the caps may be retained on the assembly 11, they are attached to the body 13, 14 or the housing 12 by means of tethers 26 and 27 which are attached to the caps and to bodies 13, 14 or housing 12, and to caps 23, and 24. Tethers 26 and 27 may be chain links, wire, or other suitable filamentary material or elongated flexible member. It is possible to cast the cap, the body, and the tether in one operation, in which case the tether is integral with cap and the body. Such tethering of the caps prevents their being misplaced or lost, thereby ensuring that the nibs will always be covered when not in use, as shown in FIG. 2.

FIGS. 3a and 3b depict a nib 28 having a sloped chisel shaped end 29 for rubbing against the surface being tested. Nib 28 is shown as having only one shaped end 29, but it is to be understood that the blank end 31 may also have a chisel or other shape, thereby doubling the utility of the nib 28 by reversing it.

FIGS. 4a and 4b depict a nib 32 having rounded ends 33 and 34. As discussed hereinbefore, it is not necessary to have both ends shaped.

FIGS. 5a and 5b show a nib 36 having a tetrahedral shaped end 37 and a blank end 38. If desired, end 38 may also be shaped having any of a number of configurations including tetrahedral.

FIGS. 6a and 6b depict a nib 39 having a pyramidal shaped end 41. Both the ends 37 of the nib 36 and the end 41 of nib 39 provide four sides which may be separately rubbed against the surface being tested, thereby being usable four times instead of the once or twice of the prior art.

The numerous enumerated features of construction may be modified or otherwise altered by workers in the art, it being intended that such changes fall within the scope of the present invention as claimed.

Therefore, having thus described the invention, at least the following is claimed:

1. A paint testing apparatus for determining the type of paint used on a surface to be treated, said apparatus comprising:
an elongated housing being open at both ends;
a substantially hollow body member inserted into said housing at each end thereof in a press fit;
first and second porous nibs being mounted in a tight slip fit in the body members at each end of the housing and exposed to the hollow interior of said housing;
said first and second nibs having contrasting light and dark colors;
a paint solvent within said housing in contact with at least one of said nibs for saturating it; and
nib protective caps at each end of the apparatus for enclosing the nibs so saturated.

2. The paint testing apparatus as claimed in claim 1 wherein at least one of said nibs has a chisel shaped configured end.

3. The paint testing apparatus as claimed in claim 1 wherein at least one of said nibs has a rounded end configuration.

4. The paint testing apparatus as claimed in claim 1 wherein at least one of said nibs has a tetrahedral shaped end configuration.

5. The paint testing apparatus as claimed in claim 1 wherein at least one of said nibs has an enlarged four sided pyramidal configuration at one end thereof.

6. The paint testing apparatus as claimed in claim 1 wherein at least one of said nibs has first and second configured ends.

7. The paint testing apparatus as claimed in claim 1 wherein at least one of said nibs is made of a material comprising a combination of a polyester fiber and a resin.

8. The paint testing apparatus as claimed in claim 1 wherein said paint solvent comprises 2-butoxyethanol or ethylene glycol monobutyl ether to impart extended shelf life to the apparatus.

9. The paint testing apparatus as claimed in claim 1 wherein each of said body members has a tapered nose portion for receiving a nib in a tight slip fit.

10. The paint testing apparatus as claimed in claim 9 wherein each of said caps has a tapered configuration matching the taper of the nose portion for mounting the cap to the nose portion by which it is held in position enclosing the corresponding nib.

11. The paint testing apparatus as claimed in claim 1 wherein each of said caps is tethered to the apparatus by means of an elongated flexible member which is affixed to said cap and the apparatus.

12. The paint testing apparatus as claimed in claim 11 wherein said elongated flexible member is integral with the cap and the apparatus.
